(19) United States
(12) Patent Application Publication
(10) Pub. No.: US 2020/0202999 A1
(43) Pub. Date: Jun. 25, 2020

(54) DATA PROCESSING APPARATUS, MAGNETIC RESONANCE IMAGING APPARATUS AND MACHINE LEARNING APPARATUS

(71) Applicant: Canon Medical Systems Corporation, Otawara-shi (JP)

(72) Inventor: Hidenori TAKESHIMA, Kawasaki (JP)

(73) Assignee: Canon Medical Systems Corporation, Otawara-shi (JP)

(21) Appl. No.: 16/697,269

(22) Filed: Nov. 27, 2019

(30) Foreign Application Priority Data
Dec. 20, 2018 (JP) 2018-238475

(51) Int. Cl. G16H 30/20; G06N 3/08

(52) U.S. Cl. CPC G16H 30/20; G06N 3/08

(57) ABSTRACT

According to one embodiment, a data processing apparatus includes processing circuitry. The processing circuitry is configured to group a plurality of channels of input data based on a physical relationship between the input data to classify the plurality of channels into a plurality of subsets. The processing circuitry is configured to perform convolutional processing of the input data in units of subsets for the plurality of subsets.

### CROSS-REFERENCE TO RELATED APPLICATIONS

This application is based upon and claims the benefit of priority from the prior Japanese Patent Application No. 2018-238475, filed Dec. 20, 2018, the entire contents of which are incorporated herein by reference.

### FIELD

Embodiments described herein relate generally to a data processing apparatus, a magnetic resonance imaging apparatus and a machine learning apparatus.

### BACKGROUND

In a convolutional neural network (CNN) in general, if there are 100 channels as inputs, all of them are subjected to each convolutional processing and 100 channels in total are output. In other words, even if 100 channels are output for the input 100 channels, all input channels are mixed in each output data.

In this convolutional processing, the relative position of the input channels is ignored in the process of training. Furthermore, depending on the relationship between input channels, a relatively useless channel, which is not effective for the convolutional processing, may exist. However, under present circumstances, even the channels that are considered useless are subjected training, and the training is performed to eliminate useless channels by selection.

Therefore, many useless coefficients occur during the training. Thus, the problems of high costs and consumption of much time are involved in the training of models, and the outputs obtained from the trained model are low in accuracy.

### BRIEF DESCRIPTION OF THE DRAWINGS

FIG. 1 is a conceptual diagram showing a data processing system according to a first embodiment.

FIG. 2 is a block diagram showing a data processing system according to the first embodiment.

FIG. 3 is a diagram showing an example of data input and output for a CNN according to the first embodiment.

FIG. 4 is a diagram showing details of data input in and output from a convolutional layer according to the first embodiment.

FIG. 5 is a diagram showing an example of generation of additional channel subsets according to a first modification of the first embodiment.

FIG. 6 is a diagram showing an example of generation of additional channel subsets according to a second modification of the first embodiment.

FIG. 7 is a diagram showing an example of generation of a channel subset according to a third modification of the first embodiment.

FIG. 8 is a diagram showing an example of generation of a channel subset according to a fourth modification of the first embodiment.

FIG. 9 is a diagram showing an example of generation of a channel subset according to a fifth modification of the first embodiment.

FIG. 10 is a diagram showing an example of generation of a channel subset in a residual network (ResNet) according to a second embodiment.

FIG. 11 is a diagram showing an example of generation of a channel subset in a densely connected convolutional network (DenseNet) according to the second embodiment.

FIG. 12 is a diagram showing a configuration of a magnetic resonance imaging (MRI) apparatus according to a third embodiment.

FIG. 13 is a diagram showing an example of generation of a channel subset in a case of using an MR image as input data according to the third embodiment.

### DETAILED DESCRIPTION

In general, according to one embodiment, a data processing apparatus includes processing circuitry. The processing circuitry is configured to group a plurality of channels of input data based on a physical relationship between the input data to classify the plurality of channels into a plurality of subsets. The processing circuitry is configured to perform convolutional processing of the input data in units of subsets for the plurality of subsets.

In the following descriptions, a data processing apparatus, a magnetic resonance imaging apparatus (MRI apparatus), a machine leaning apparatus, and a machine learning method according to the embodiments of the present application will be described with reference to the drawings. In the embodiments described below, elements assigned with the same reference symbols are assumed to perform the same operations, and redundant descriptions thereof will be omitted as appropriate. Hereinafter, an embodiment will be described with reference to the accompanying drawings.

### First Embodiment

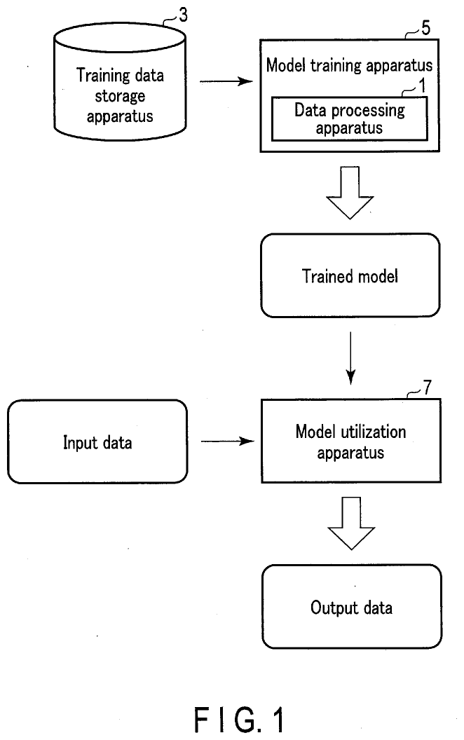

In the first embodiment, a trained machine-learning model (hereinafter referred to as the trained model) is assumed. A conceptual diagram of a data processing system, showing a flow of generation and use of the trained model will be described with reference to FIG. 1.

The data processing system includes a training data storage apparatus 3, a model training apparatus 5 including a data processing apparatus 1, and a model utilization apparatus 7.

The training data storage apparatus 3 stores training data including a plurality of training samples. The training data storage apparatus 3 is, for example, a computer or a workstation with a large-capacity storage incorporated therein. Alternatively, the training data storage apparatus 3 may be a large-capacity storage communicably connected to a computer via a cable or a communication network. As such a storage, a hard disk drive (HDD), a solid state drive (SSD), or an integrated circuit storage, etc. can be used as appropriate.

The model training apparatus 5 generates a trained model by training a machine learning model using the data processing apparatus 1 based on training data stored in the training data storage apparatus 3 according to a model training program. The model training apparatus 5 is a computer such as a workstation, including a processor, for example, a central processing unit (CPU), a graphics processing unit (GPU), etc. Details of the model training apparatus 5 will be described later.

The machine learning model of the present embodiment is assumed to be a convolutional neural network (CNN) including a convolutional layer. However, any other machine learning model including convolutional processing is applicable to the present embodiment.

The model training apparatus 5 and the training data storage apparatus 3 may be communicably connected via a cable or a communication network, or the training data storage apparatus 3 may be included in the model training apparatus 5. In such a case, training data is supplied from the training data storage apparatus 3 to the model training apparatus 5 via the cable or the communication network.

The model training apparatus 5 and the training data storage apparatus 3 need not be communicably connected. In such a case, training data is supplied from the training data storage apparatus 3 to the model training apparatus 5, via a portable storage medium storing the training data thereon.

The model utilization apparatus 7 generates output data corresponding to the input data to be processed, using the trained model obtained through training by the model training apparatus 5 in accordance with the model training program. The model utilization apparatus 7 may be, for example, a computer, a workstation, a tablet PC, a smartphone, or an apparatus for use in specific processing, such as a medical diagnosis apparatus. The model utilization apparatus 7 and the model training apparatus 5 may be communicably connected via a cable or a communication network. In such a case, the trained model is supplied from the model training apparatus 5 to the model utilization apparatus 7 via the cable or the communication network. The model utilization apparatus 7 and the model training apparatus 5 are not necessarily communicably connected. In such a case, the trained model is supplied from the model training apparatus 5 to the model utilization apparatus 7 via a portable storage medium storing the trained model thereon.

In the data processing system shown in FIG. 1, the training data storage apparatus 3, the model training apparatus 5 and the model utilization apparatus 7 are depicted as separate apparatuses. However, those apparatuses may be constructed as one integral unit.

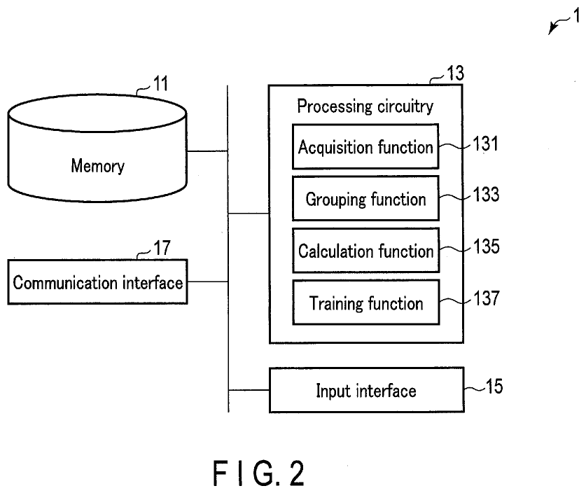

An example of the data processing apparatus 1 according to the first embodiment will be explained with reference to the block diagram of FIG. 2.

The data processing apparatus 1 includes a memory 11, processing circuitry 13, an input interface 15, and a communication interface 17. The memory 11, the input interface 15, and the communication interface 17 may be contained in the model training apparatus 5, not the data processing apparatus 1, or may be shared by the data processing apparatus 1 and the model training apparatus 5.

The memory 11 is a storage, such as a read only memory (ROM), a random access memory (RAM), an HDD, an SSD, or an integrated circuit storage, etc., which stores various types of information. The memory 11 stores, for example, a machine learning model and training data. The memory 11 may be not only the aforementioned storage, but also a driver that writes and reads various types of information in and from, for example, a portable storage medium such as a compact disc (CD), a digital versatile disc (DVD), or a flash memory, or a semiconductor memory.

Furthermore, the memory 11 may be located within another computer connected to the data processing apparatus 1 through a network.

The processing circuitry 13 includes an acquisition function 131, a grouping function 133, a calculation function 135, and a training function 137. First, processing when training the machine learning model will be described.

The processing circuitry 13 acquires training data from the training data storage apparatus 3 by the acquisition function 131. Input data of the training data may be any data, for example, a one dimensional time-series signal, two-dimensional image data, three-dimensional voxel data, or higher-dimensional data.

The processing circuitry 13 groups a plurality of input channels of the input data by the grouping function 133 based on the physical relationship between the input data of the training data, and generates a plurality of subsets. In the following, a subset of a plurality of input channels, which are grouped, is referred to as a channel subset. The physical relationship in this embodiment means a relationship concerning a physical quantity, such as a time, a position (coordinates), and a distance. Specifically, adjacent channels having a close physical relationship between input data are considered as being close. For example, if the input data is a moving picture, images of consecutive frame numbers constituting the moving picture are output at close time intervals. If the input data is a medical image, images of consecutive slice positions (slice numbers) have close captured time and close space coordinates.

The processing circuitry 13 performs convolutional processing of the input data in units of channel subsets by the calculation function 135 for a plurality of channel subsets. The processing circuitry 13 performs training of the machine learning model using the training data by the training function 137, so that output data of the training data can be output. The trained model is thus generated. Next, processing when utilizing the trained model will be described.

The processing circuitry 13 acquires data to be processed by the acquisition function 131.

The processing circuitry 13 groups a plurality of input channels of the data to be processed by the grouping function 133 based on the physical relationship between the data to be processed, and generates a channel subset.

The processing circuitry 13 applies the trained model to the channel subsets, so that convolutional processing of the data to be processed can be performed in units of channel subsets for the plurality of channel subsets by the calculation function 135.

The input interface 15 receives various types of input operations from a user, converts the received input operations to electric signals, and outputs the electric signals to the processing circuitry 13. Specifically, the input interface 15 is connected to an input device, such as a mouse, a keyboard, a trackball, a switch, a button, a joystick, a touch pad, or a touch panel display. The input interface 15 outputs the electric signals corresponding to the input operations received by the input device to the processing circuitry 13. The input device connected to the input interface 15 may be an input device provided on another computer and connected via a network or the like.

The communication interface 17 is an interface for data communication with, for example, the model training apparatus 5, the training data storage apparatus 3, or another computer.

In the data processing apparatus 1, the memory 11 may store training data and the processing circuitry 13 may include a training function for training of a machine learning model in the same manner as in the model training apparatus 5. As a result, the data processing apparatus 1 alone may perform training of the machine learning model including the convolutional layer and generate the trained model.

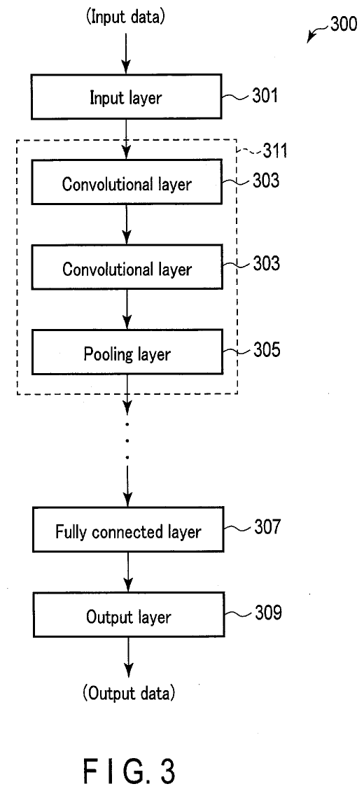

An example of the data input and output for the CNN according to the first embodiment will now be explained with reference to FIG. 3.

As shown in FIG. 3, the CNN 300 assumed in the first embodiment includes an input layer 301, a convolutional layer 303, a pooling layer 305, a fully connected layer 307, and an output layer 309. FIG. 3 shows an example, in which a plurality of processing blocks 311, each including the pooling layer 305 after two convolutional layers 303, are arranged before the fully connected layer 307. The embodiment is not limited to the processing blocks 311 described above. The number and order of the convolutional layers 303 and the pooling layers 305 may be set as appropriate.

Input data is supplied to the input layer 301. It is assumed that input data is a set of values. The input data may be arranged and read out as a set of input data (as one channel) into the memory, or as a set of elements of input data into the memory.

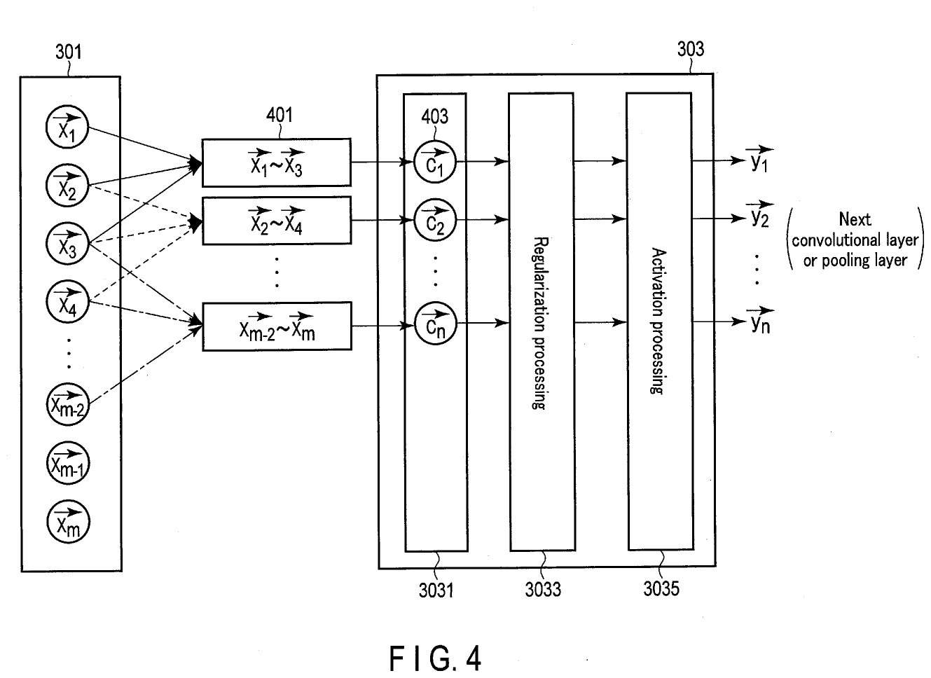

In the convolutional layer 303, convolutional processing is performed for input data from the input layer 301. Details of the convolutional processing will be described later with reference to FIG. 4.

In the pooling layer 305, for example, max pooling processing is performed for the convolutional-processed data. Since general processing is performed in the pooling layer 305, a detailed description of the processing is omitted.

In the fully connected layer 307, the data processed in the processing block 311 and channels in the fully-connected layer 307 are fully-interconnected between layers.

In the output layer 309, for example, the softmax function is applied to an output from the fully connected layer 307, and output data, which is a final output from the trained model, is generated. The function in the output layer is not limited to the softmax function, and any other function may be selected in accordance with an output format desired by the CNN 300. For example, if the CNN 300 is used for binary classification, a logistic function may be used. If the CNN 300 is used for a regression problem, linear mapping may be used.

Details of data input and output in the convolutional layer 303 according to the first embodiment will now be described with reference to FIG. 4. FIG. 4 shows an interlayer connection between the input layer 301 and the convolutional layer 303. Since the input data is vector data, a channel x is expressed as a vector in the figure.

In the input layer 301, m channels x₁ to xₘ of input data are given. It is assumed that the input data of adjacent channels have a consecutive (or close) physical relationship with each other. However, the embodiment is not limited to this assumption.

For example, the input data of the consecutive physical relationship may have discontinuous channel numbers. For example, time-series input data #1 to #5 are not necessarily set as channels x₁ to x₅, and may be set as channels x₁, x₂, x₁₀, x₅, and x₈ of discontinuous channel numbers.

In this case, the memory 11 prestores a look-up table indicating the correspondence between a physical relationship of input data and a channel number. When generating a channel subset, the processing circuitry 13 may refer to the look-up table by the grouping function 133, and select channels of data having a close physical relationship of input data as the channel subset.

The convolutional layer 303 includes convolutional processing 3031, regularization processing 3033, and activation processing 3035. The regularization processing 3033 and the activation processing 3035 are not essential, and may be adopted as needed in accordance with implementation.

The processing circuitry 13 groups a plurality of channels of the data having a close physical relationship by the grouping function 133, and generates a plurality of channel subsets 401. In other words, the channel subsets 401 are generated by grouping channels based on given physical conditions. The blocks of the channel subsets 401 shown in FIG. 4 are depicted for convenience of explanation to explain the combination of the channels in the input layer 301 that are grouped into the channel subsets 401, and do not represent new layers connected to the input layer 301.

In the example shown in FIG. 4, three adjacent channels, i.e., the channels x₁ to x₃, the channels x₂ to x₄, and the channels xₘ₋₂ to xₘ are grouped to generate the channel subsets 401.

In the convolutional processing 3031, the channel x in the input layer 301 is subjected to convolutional processing in units of channel subsets. In the convolutional processing 3031, convolutional processing with a kernel is performed in units of channel subsets, and the convolutionally-processed data (hereinafter also referred to as a feature map) are generated.

Specifically, the feature map c₁ is generated by convolutionally processing the three adjacent channels of the channels x₁ to x₃ with a kernel. Similarly, the feature map c₂ is generated by convolutionally processing the three adjacent channels of the channels x₂ to x₄, which are shifted by one channel from the channels x₁ to x₃, with the kernel. In the example of FIG. 4, as a result, n feature maps cₙ are generated (n is a positive integer that satisfies m>n).

FIG. 4 shows convolutional processing with one kernel. However, since the convolutional processing uses a plurality of kernels, a plurality of feature maps is generated by convolutional processing of one channel subset and each of the kernels. In other words, feature maps of the same number as the number of kernels are generated from one channel subset.

The combination of the channels constituting the channel subsets 401 may be determined by manually selecting channels of the input layer 301 by the user, or may be automatically determined.

In the case of manually determining the channel subsets 401, the processing circuitry 13 may receive user's instructions via the interface, and group a plurality of channels in accordance with the user's instructions by the grouping function 133, thereby generating the channel subsets 401.

In the case of automatically determining the channel subsets 401, if the physical relationship of input data depends on the time-series order, the processing circuitry 13 may generate the channel subsets 401 by grouping a plurality of input data items obtained in a given period of time by the grouping function 133. Alternatively, for example, it is assumed that the data processing apparatus 1 of the first embodiment estimates a P wave generated when an earthquake occurs. In this case, if earthquake waves observed at the respective observation points are input to the processing circuitry 13, the processing circuitry 13 may divide an area in accordance with distances from a point assumed to be an earthquake center, and may generate the channel subsets 401 by grouping the earthquake waves observed at the respective points that are geographically close to one another. In this case, the physical relationship in the grouping depends on the distances.

The channel subsets 401 may be determined by using so-called L1 optimization, which means searching for an optimized solution by Lasso regression using L1 regularization. Furthermore, using as training data the input data and the channel subset 401, which is a correct solution, the channel subsets 401 may be determined by using the trained model, which has been trained to output the channel subsets 401 from the input data.

FIG. 4 shows an example of grouping the channels so that the number of channels that do not overlap between the adjacent channel subsets 401 is one; that is, the channels forming the adjacent channel subsets are shifted by one. However, the embodiment is not limited to this example, and the number of channels that do not overlap between the adjacent channel subsets 401 may be greater. For example, 16 channels of the channel x₁ to the channel x₁₆ may be grouped as a channel subset 401, and 16 channels of the channel x₉ to the channel x₂₅ may be grouped as another channel subset 401. Thus, the number of channels that do not overlap may be more than one.

In other words, the number of channels that are grouped into a channel subset and the number of overlapping channels between the adjacent channel subsets 401 may be suitably determined in accordance with the given physical relationship.

In the regularization processing 3033, the feature maps obtained by the convolutional processing 3031 are input, and batch normalization processing is performed. Since general processing can be adopted as the batch normalization processing, detailed description of the processing is omitted.

In the activation processing 3035, for example, an activation function, such as a rectified linear unit (ReLU), is applied to the feature maps after the batch normalization processing by the regularization processing 3033, and n data y₁ to yₙ to be finally output from the convolutional layer are generated.

The data y₁ to yₙ are input to the adjacent convolutional layer 303 or the adjacent pooling layer 305 of the lower layer.

In the convolutional processing of the present embodiment as shown in FIG. 4, the convolutional processing is not performed for all of the channels x₁ to xₘ of the input data, unlike the convolutional processing performed in general convolutional neural networks, but is performed for the channel subsets based on the physical relationship between the input data, which are constituted by a smaller number of channels than all channels. Accordingly, the amount of calculation and the amount of memory required for the convolutional processing can be noticeably reduced.

### First Modification of First Embodiment

In the first embodiment described above, since the outputs from the channel subsets 401 and the outputs from the convolutional layer 303 coincide in one-to-one correspondence, the number n of channels of the data output from the convolutional layer 303 is less than the number m of channels in the input layer 301. For example, if the number of channels in the input layer 301 is 16 and the channel subsets 401 are generated using three channels, 14 channel subsets 401 are formed. Therefore, the number of channels of the data output from the convolutional layer 303 is 14. The number of channels of the data output from the convolutional layer 303 may be less than the number of channels of the data input to the convolutional layer 303. However, in some cases to which the trained model is applied, it is preferable that the number of channels of input data to the convolutional layer and the number of channels of output data should be the same.

Therefore, in addition to the channel subsets 401, additional channel subsets may be generated, each containing less channels than those grouped as the channel subsets 401 and formed of a combination different from those of the channel subsets 401.

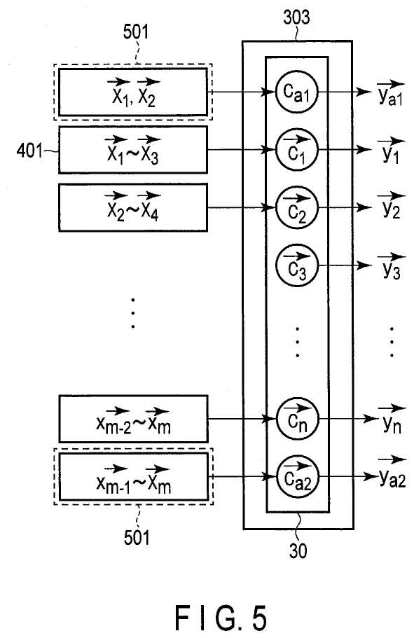

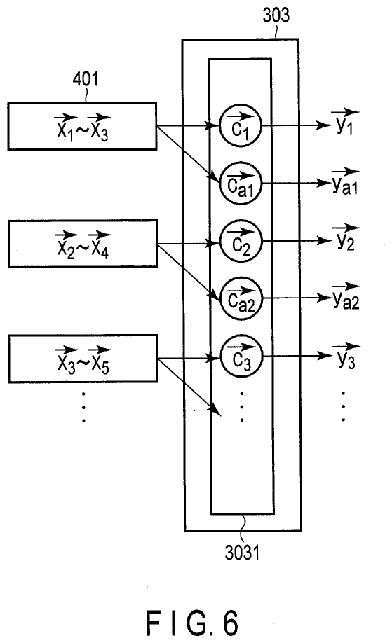

An example of generation of the additional channel subsets according to the first modification of the first embodiment will be explained with reference to FIG. 5.

FIG. 5 shows the channel subsets 401 of the input data and the convolutional layer 303. Since the regularization processing 3033 and the activation processing 3035 of the convolutional layer 303 are the same as those in the first embodiment, only the convolutional processing 3031 will be described. In the example of FIG. 5, in the same manner as in the example of FIG. 4, a channel subset 401 is formed of three channels; that is, a channel subset 401 formed of channels x₁ to x₂ to a channel subset 401 formed of channels xₘ₋₂ to xₘ are generated.

By the grouping function 133, the processing circuitry 13 groups two channels (x₁ and x₂), which is less than the three channels forming the channel subset 401, and generates an additional channel subset 501. Similarly, the processing circuitry 13 groups two channels (xₘ₋₁ and xₘ), and generates another additional channel subset 501. Feature maps Cal and Cat are generated from the additional channel subsets 501; as a result, output channels yₐ₁ and yₐ₂ are obtained. As a result, the number of channels of data input to the convolutional layer 303 and the number of channels of data output from the convolutional layer 303 can be the same. Furthermore, the number of additional channel subsets 501 may be further increased, so that the number of channels of output data can be greater than the number of channels of input data.

If the input data are time-series data, channel positions of input data used as the additional channel subsets 501 are assumed to be end portions (a first portion and a last portion of the channel numbers), namely, the channels x₁ and x₂ and the channels xₘ and xₘ₋₁, which are less frequently selected (grouped) as channel subsets.

However, additional channel subsets 501 can be generated not only at the end portions of the channels but at any channel positions. Specifically, if half of m is p, processing to set channels xₚ and xₚ₊₁ as an additional channel subset may be performed.

According to the first modification of the first embodiment described above, the number of channels output from the convolutional layer can be increased.

### Second Modification of First Embodiment

As a method different from the first modification, convolutional processing may be performed a plurality of times for the same channel subset to increase the number of outputs.

An example of generation of additional channel subsets according to the second modification of the first embodiment will be explained. The processing circuitry 13 selects a channel subset to be subjected to the convolutional processing a plurality of times by the grouping function 133. The processing circuitry 13 performs the convolutional processing for the selected channel subset a plurality of times by the calculation function 135.

Specifically, by the calculation function 135, the processing circuitry 13 performs first convolutional processing for the channel subset 401 formed of, for example, channels x₁-x₃, and obtains an output channel y₁ as a processing result from the convolutional layer 303. Then, the processing circuitry 13 performs second convolutional processing for the same channel subset 401, and obtains an output channel yₐ₁ as a processing result. As a result, two outputs can be obtained from one channel subset 401, and the two outputs are output to a lower layer. The channel subsets 401 to be subjected to convolutional processing a plurality of times may be selected as appropriate in accordance with the design; for example, channel subsets at end portions may be selected. Furthermore, the convolutional processing may be performed while changing the initial value of a weighting parameter in the convolutional processing each time.

In the case of performing the convolutional processing a plurality of times, the combination of channels to be grouped as a channel subset 401 may be slightly changed for each training. For example, a reference position (reference channel) to be a reference of grouping of channel subsets 401 may be shifted by a decimal number. Specifically, if the channel subset 401 is generated from channels x₁ to x₃ about x₂ as a reference channel by the first convolutional processing, the channel subset 401 may be generated with reference to a virtual channel x_{2.5} between channel x₂ and x₃ in the second convolutional processing. In this case, four channels x₁ to x₄ are grouped as a channel subset. Using the trained model obtained by training, in the case of image processing, for example, data for a frame that is not actually acquired can be output in the same manner as frame interpolation.

According to the second modification of the first embodiment described above, the convolutional processing is performed a plurality of times for the same channel subset, so that different data can be output in accordance with the number of times. Thus, in the same manner as in the first modification, the number of channels of output data can be increased.

### Third Modification of First Embodiment

Data used as input data may be obtained from different information sources. For example, different kinds of data obtained from different medical diagnosis apparatuses may be used. Specifically, it is assumed that an Electrocardiogram (ECG) signal acquired from an electrocardiographic inspection apparatus and an MR image acquired from an MRI apparatus are used as input data for training. In this case, for example, in one second, about 1000 samples of data are obtained from the ECG signals. On the other hand, since it takes more time to acquire an MR image as compared to an ECG signal, only about 100 samples of data may be obtained from the MR images in one second. Thus, the numbers of samples of the two kinds of data acquired in a fixed period considerably differ.

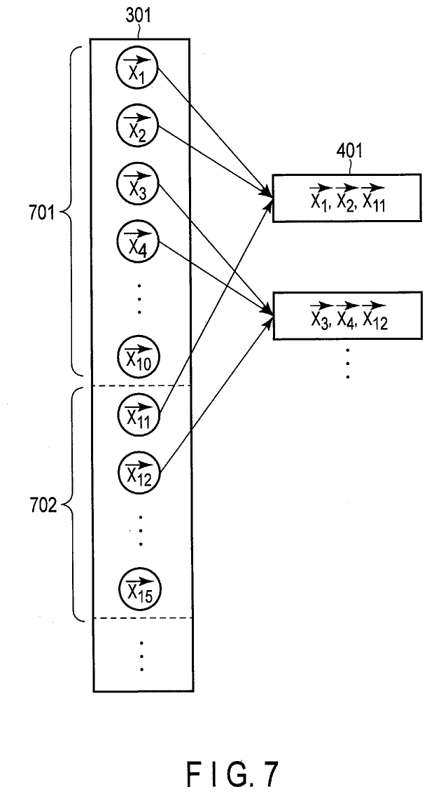

An example of generation of channel subsets in the case of using different kinds of data as input data will be explained with reference to FIG. 7.

FIG. 7 shows an example of using ECG signals and MR signals as input data. In this case, different kinds of input data of different numbers of samples are input as channels of the input layer 301. For example, a first input data set 701 is formed of ECG signals, and set as input channels x₁ to x₁₀ of the input layer 301. A second input data set 702 is formed of MR images, and set as input channels x₁₁ to x₁₅ of the input layer 301.

If the first input data set 701 and the second input data set 702 are time-series data, the processing circuitry 13 selects, by the grouping function 133, data acquired as having a physical relationship of input data in the same period from the first input data set 701 and the second input data set 702, thereby generating a channel subset 401. Specifically, the number of samples of the first input data set 701 is twice the number of samples of the second input data set 702. Therefore, the channels x₁ and x₂ of the first input data set and the channel x₁₁ of the second input data set are grouped to generate the channel subset 401.

It is assumed that the first input data set 701 and the second input data set 702 are configured so that data in the same period of the input data sets that are synchronized in time series are selected as a channel subset. However, the channel subset 401 may be generated intentionally from the input data sets that are not in synchronism. By training of the machine learning model by the grouped channel subsets using the input data that are not synchronized, even if data that are not synchronized in different input data sets are processed, robust execution results can be obtained.

If the method of increasing the number of channels of output data according to the first modification is applied to the third modification, it is not necessary to set the number of additional channel subsets in accordance with the ratio of data sets. In other words, if the number of the first input data sets 701 is twice the number of second input data sets 702 and the number of additional channel subsets added to the first input data sets 701 is 20, the number of additional channel subsets added to the second input data sets 702 is not necessarily 10, but may be suitably increased or decreased.

Although the example of using data acquired from different information sources (apparatuses) as different kinds of data is described above, similar processing can be performed in the case of using different kinds of data acquired in the same apparatus. For example, images acquired in a high resolution mode and images acquired in a low resolution mode, which is half that of the high resolution mode, may be used as input data. In this case, since the number of channels of the data of the images acquired in the high resolution mode is twice that of the images acquired in the low resolution mode, the method of generating channel subsets of the third modification described above is applicable.

According to the third modification of the first embodiment, even if different kinds of data are input, convolutional processing can be performed in consideration of the physical relationship between the input data.

### Fourth Modification of First Embodiment

In the first embodiment and the modifications described above, the channel subsets are generated so as to have data locality in consideration of the physical relationship between channels. In the fourth embodiment, globality relating to the overall channels are further considered, while considering the locality mentioned above.

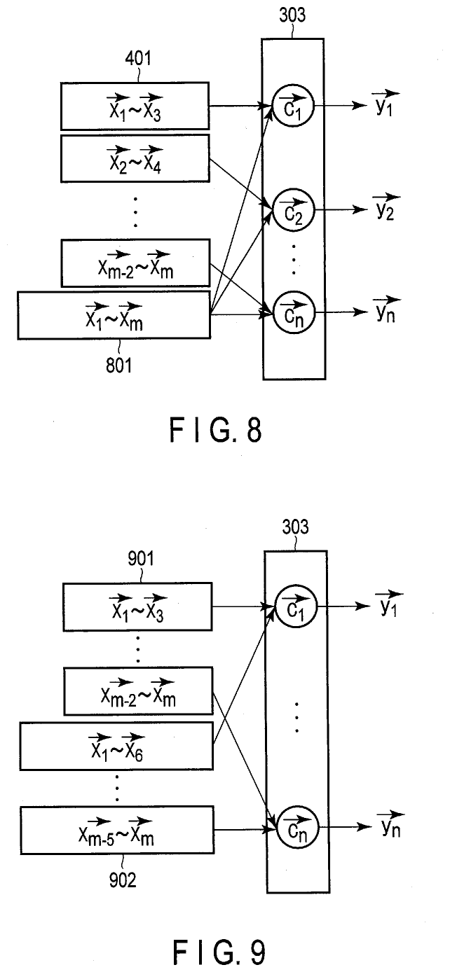

An example of generation of the channel subsets according to the fourth modification will be explained with reference to FIG. 8.

In the example shown in FIG. 8, all channels 801 (x₁ to xₘ), in addition to the channel subsets 401 shown in FIG. 4, are input to the convolutional layer 303. In particular, the convolutional processing 3031 convolutes each of the channel subsets 404 with all channels 801 (x₁ to xₘ).

According to the fourth modification described above, through the convolutional processing of each of the channel subsets with all channels, the local physical relationship can be trained by the channel subsets, while the bias component applied to the entire body of input data can be removed. In particular, it is possible to remove long-term variations of low-frequency components of images or sound signals that cannot be removed by a simple bias.

### Fifth Modification of First Embodiment

In the fifth modification, a plurality of kinds of channel subsets having different numbers of data items are generated from the same input data.

An example of generation of the additional channel subsets according to the fifth modification will be explained with reference to FIG. 9.

The processing circuitry 13 generates, by the grouping function 133, first channel subsets 901, each obtained by grouping three consecutive channels of the input data, and second channel subsets 902, each obtained by grouping a greater number of first channel subsets 901, in the present case, six channels of the input data.

The processing circuitry 13 performs, by the calculation function 135, convolutional processing of data based on the physical relationship between input data of the first channel subsets 901 and the second channel subsets 902. In the example shown in FIG. 9, the convolutional processing is performed with respect to, for example, the first channel subset 901 formed of the channels x₁ to x₃ and the second channel subset 902 formed of the channels x₁ to x₆, thereby generating a feature map c₁.

Thus, channel subsets having different numbers of data items are used to generate channel subsets of a plurality of patterns, so that the processing can be performed to deal with multi-resolution in consideration of a plurality of physical relationships from one input data item.

Even if channel subsets have the same number of data items, if the channel subsets are formed of different channels, the same effect as that of the fifth modification can be obtained. In other words, a channel subset may be generated by selecting discrete channels within a range of channels having a physical relationship that satisfies a specific condition, such as the channels in a fixed period, instead of selecting channels that are in consecutive order in physical relationship. Specifically, the convolutional processing is performed with respect to the first channel subset of the channels x₁ to x₃ and the second channel subset of the channels x₁, x₄, and x₆.

In this case, since the second channel subset includes the channels x₁ and x₆, it may have a tendency of the physical relationship of the channels x₁ to x₆.

In the case of processing periodic signals or the like, for example, measuring noise generated from a power supply of, for example, 50 Hz, a channel subset may be generated by grouping non-consecutive channels, that is, every several channels, instead of by selecting consecutive channels. As a result, channel subsets that are based on the properties of the periodic signals can be generated.

When training the machine learning model having the convolutional layer exemplified in the first embodiment and the modifications thereof, the machine learning model may be trained by generating channel subsets from input data of the training data and inputting the channel subsets to the convolutional layer, and a trained model may be generated from correct data for the training data output from the convolutional layer.

When utilizing the trained model, the processing circuitry 13 may input, by the calculation function 135, the channel subsets of input data to be processed into the trained model, and obtain output data based on the trained model.

The trained model of the first embodiment is applicable to processing in which the CNN is used; for example, image recognition, image identification, image correction, speech recognition, estimation of R waves of ECG, denoising, automated driving, genome analysis, abnormality detection, etc.

According to the first embodiment described above, the convolutional processing is performed with respect to the channel subsets in consideration of the physical relationship of input data, so that training in consideration of the locality of the channels can be performed and the number of parameters to be trained can be greatly reduced. Therefore, the amount of calculation and the amount of required memory can be reduced.

When utilizing the trained model, the amount of calculation and the amount of required memory can be reduced as in the case of training. In addition, since data remotely related in the physical relationship are not used in the convolutional processing, occurrence of undesired noise is prevented due to the structure of training, not as a result of the training.

### Second Embodiment

The method of generating channel subsets of the first embodiment and the modifications of the first embodiment are applicable to not only a plane CNN but also a special multilayered CNN, such as a residual network (ResNet) and a densely connected convolutional network (DenseNet).

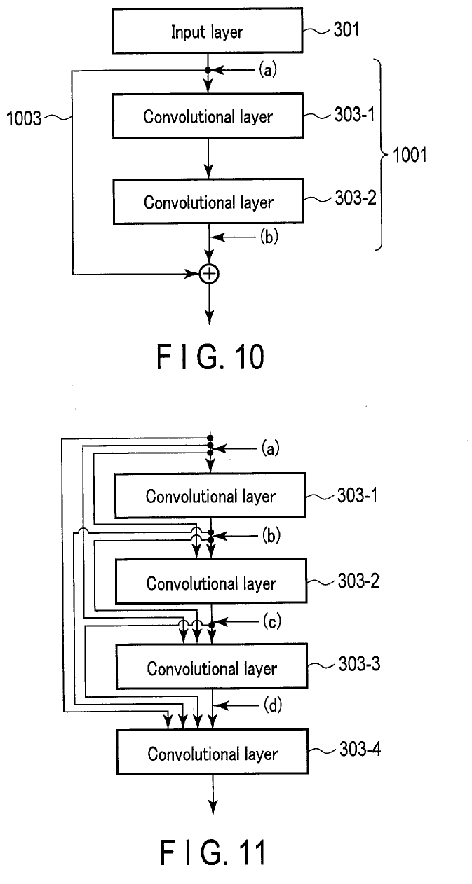

An example of generation of channel subsets in the ResNet will be explained with reference to FIG. 10.

FIG. 10 shows a concept of a residual block in the ResNet.

The residual block includes a route 1001 connecting a plurality of convolution layers 303, and a route 1003 connecting inputs of the convolutional layers 303 to an output diverting around the convolutional layers 303. The ResNet is formed of a plurality of residual blocks as mentioned above. The activation processing, for example, ReLU (not shown) of the last convolutional layer in the residual block may be provided after connection of the input and the output.

To apply the design method of the channel subsets of the first embodiment and the modifications thereof, the number of channels of data output from the last convolutional layer 303-2 of the residual block may be the same as the number of channels of data input to the residual block. Specifically, the processing circuit 13 may increase, by the grouping function 133, the number of channels of data output from the convolutional layer 303-2 using, for example, the first modification or the second modification of the first embodiment, so that the number of channels of data input to the convolutional layers ((a) in FIG. 10) becomes the same as the number of channels of data output from the convolutional layer 303-2 ((b) in FIG. 10).

Next, an example of generation of channel subsets in the DenseNet will be explained with reference to FIG. 11.

FIG. 11 shows a concept of a dense block in a DenseNet.

In the dense block, an initial input and outputs of all preceding convolutional layers are input to a convolutional layer. In the example shown in FIG. 11, the input to the convolutional layer 303-1 and three outputs of the convolutional layers 303-1 to 303-3 are input to the last convolutional layer 303-4; that is, the number of inputs to the last convolutional layer 303-4 is four.

If the number of channels of data input to the convolutional layer 303-1 is 32 and the number of channels of data output from the convolutional layer 303-1 is also 32, the number of channels of data input to the convolutional layer 303-4 represented by (d) in FIG. 11 is 32×4=128.

Therefore, channels #1 to #5 output from the convolutional layers 303-1 to 303-3 are combined and input to the convolutional layer 303-4. That is, input data of M channels corresponding to the input channels #1 to #5 (namely 5×M channels) are input to the M-th convolutional layer.

To apply the method of generating the channel subsets according to the first embodiment and the modifications thereof, if the channel subsets are generated from inputs of M channels, interleave may be performed to maintain the physical relationship. For example, the channels input to the convolutional layer 303-3 indicated by (c) in FIG. 11 are channels #1 to #5 corresponding to input data to the dense block, channels #1 to #5 output from the convolutional layer 303-1, and channels #1 to #5 output from the convolutional layer 303-2. Each channel includes three data items.

Therefore, when the data are input to the convolutional layer 303-3, the processing circuitry 13, by the grouping function 133, generates channel subsets so that the channels are rearranged to “#1, #1, #1, #2, #2, #2, . . . .”

Alternatively, the data input to the convolutional layer are sequentially set as channels without interleaving, and the channels that have the corresponding physical relationship of data may be selected.

Specifically, for example, the channels #1 to #5 corresponding to data input to the dense block, the channels #1 to #5 output from the convolutional layer 303-1, and the channels #1 to #5 output from the convolutional layer 303-2 are sequentially arranged in the order to be input channels of the convolutional layer 303-3. Thus, the data in the convolutional layer 303-3 are set to be “#1, #2, #3, #4, #5, #1, #2, . . . ” as the input channels of the convolutional layer 303-3.

The processing circuitry 13 generates the channel subsets by the grouping function 133, so that the channels of the same channel number #1, that is, the first, sixth, and elevenths channels input to the convolutional layer 303-3 can be grouped as the channel subset.

According to the second embodiment described above, the generation and the convolutional processing of the channel subsets of the first embodiment can be applied also to the special multilayer CNN configuration, such as the ResNet or the DenseNet. In the same manner as in the first embodiment, occurrence of undesired noise is prevented due to the structure of training, while the amount of calculation and the amount of required memory are reduced.

### Third Embodiment

As the third embodiment, an MRI apparatus that executes image processing with use of the trained model for imaged MR images will be explained as an example of the model utilization apparatus 7 to which the trained model of the above embodiments is applied.

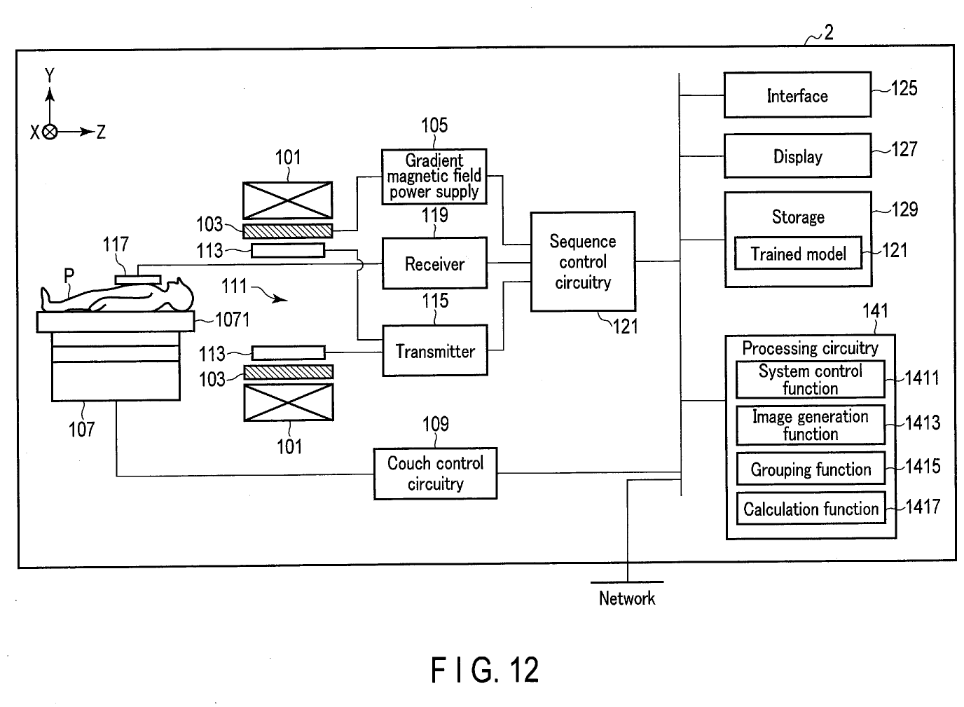

An entire configuration of an MRI apparatus 2 in the present embodiment will be described with reference to FIG. 12. FIG. 12 is a diagram showing the configuration of the MRI apparatus 2 in the present embodiment. As shown in FIG. 12, the MRI apparatus 2 includes a static field magnet 101, a gradient coil 103, a gradient magnetic field power supply 105, a couch 107, couch control circuitry 109, a transmitting coil 113, a transmitter 115, a receiving coil 117, a receiver 119, sequence control circuitry 121, a bus 123, an interface 125, a display 127, a storage 129, and processing circuitry 141. Note that the MRI apparatus 2 may include a hollow cylindrical shim coil between the static field magnet 101 and the gradient coil 103.

The static field magnet 101 is a magnet formed into a hollow approximately cylindrical shape. Note that the static field magnet 101 is not necessarily in an approximately cylindrical shape; it may be formed in an open shape. The static field magnet 101 generates a uniform static magnetic field in an internal space. For example, a superconducting magnet or the like is used as the static field magnet 101.

The gradient coil 103 is a coil formed into a hollow cylindrical shape. The gradient coil 103 is arranged inside the static field magnet 101. The gradient coil 103 is a combination of three coils corresponding to X, Y, Z-axes orthogonal to one another. The Z-axis direction is defined as the same as the direction of the static magnetic field. In addition, the Y-axis direction is defined as a vertical direction, and the X-axis direction is defined as a direction perpendicular to the Z-axis and the Y-axis. The three coils in the gradient coil 103 individually receive current supply from the gradient magnetic field power supply 105, and generate a gradient magnetic field where magnetic field strength changes along the respective axes X, Y and Z.

The gradient magnetic fields of the individual axes X, Y and Z generated by the gradient coil 103 generate, for example, the gradient magnetic field for frequency encoding (also referred to as a readout gradient magnetic field), the gradient magnetic field for phase encoding, and the gradient magnetic field for slice selection. The slice selection gradient field is used to determine an imaging slice. The phase encoding gradient magnetic field is used to change the phase of a magnetic resonance (hereinafter referred to as MR) signal in accordance with the spatial position. The frequency encode gradient field is used to change the frequency of MR signals in accordance with spatial positions.

The gradient magnetic field power supply 105 is a power supply device that supplies a current to the gradient coil 103 under the control of the sequence control circuitry 121.

The couch 107 is an apparatus having a couch top 1071 on which a subject P is placed. The couch 107 inserts the couch top 1071, on which the subject P is mounted, into a bore 111 under the control by the couch control circuitry 109. The couch 107 is installed in an examination room in which the present MRI apparatus 2 is installed in such a manner that, for example, its longitudinal direction is parallel to the central axis of the static field magnet 101.

The couch control circuitry 109 is circuitry that controls the couch 107, and drives the couch 107 in response to operator's instructions via the interface 125 to move the couch top 1071 in the longitudinal direction and vertical direction.

The transmitting coil 113 is an RF coil arranged inside the gradient coil 103. The transmitting coil 113 receives supply of an RF (Radio Frequency) pulse from the transmitter 115, and generates a transmission RF wave corresponding to a high frequency magnetic field. The transmitter coil 45 may be example, a whole body coil. The whole body coil may be used as a transmitting/receiving coil. A cylindrical RF shield is provided between the whole body coil and the gradient coil 103 to magnetically separate these coils.

The transmitter 115 supplies the RF pulse corresponding to a Larmor frequency or the like to the transmitting coil 113 by the control of the sequence control circuitry 121.

The receiving coil 117 is an RF coil arranged inside the gradient coil 103. The receiving coil 117 receives an MR signal that the radio frequency magnetic field causes the subject P to emit. The receiving coil 117 outputs the received MR signal to the receiver 119. The receiving coil 117 is, for example, a coil array having one or more coil elements, typically having a plurality of coil elements. The receiving coil 117 is, for example, a phased array coil.

The receiver 119 generates a digital MR signal, which is digitized complex number data, based on the MR signal output from the receiving coil 117 under the control of the sequence control circuitry 121. Specifically, the receiver 119 performs various types of signal processing to the MR signal output from the receiving coil 117, and then performs analog/digital (A/D) conversion to the signal subjected to the various types of signal processing. The receiver 119 samples the A/D converted data. Through this processing, the receiver 119 generates MR data. The receiver 119 outputs the generated MR data to the imaging control circuitry 121.

The sequence control circuitry 121 controls the gradient magnetic field power supply 105, the transmitter 115 and the receiver 119 or the like according to an examination protocol output from the processing circuitry 141, and performs imaging on the subject P. The examination protocol has different pulse sequences in accordance with a type of examination. The imaging protocol defines the magnitude of the current supplied from the gradient magnetic field power supply 105 to the gradient coil 103, timing of the supply of the current from the gradient magnetic field power supply 105 to the gradient coil 103, the magnitude of the RF pulse supplied from the transmitter 115 to the transmitting coil 113, timing of the supply of the RF pulse from the transmitter 115 to the transmitting coil 113, and timing of reception of the MR signal at the receiving coil 117, and the like. The sequence control circuitry 121 outputs the MR data received from the receiver 119 to the processing circuitry 141.

The bus 123 is a transmission path through which data is transmitted between the interface 125, the display 127, the storage 120, and the processing circuitry 141. Various types of biological signal measuring instruments, external storages, modalities, etc. may be connected to the bus 123 via a network, etc., as needed. For example, an electrocardiograph not shown in the figure is connected to the bus, as a biological signal measuring instrument.

The interface 125 includes circuitry that receives various types of instructions and information input from the operator. The interface 125 includes a circuit relating to, for example, a pointing device such as a mouse, or an input device such as a keyboard. The circuit included in the interface 125 is not limited to a circuit relating to a physical operational component, such as a mouse or a keyboard. For example, the interface 125 may include electric signal processing circuitry that receives an electric signal corresponding to an input operation through an external input device provided separately from MRI apparatus 2 and outputs the received electric signal to various types of circuitry.

The display 127 displays various kinds of magnetic resonance images (MR images) generated by an image processing function 1413 and various kinds of information relating to imaging and image processing, under the control by a system control function 1411 in the processing circuitry 141. The display 127 is a display device, for example, a CRT display, a liquid crystal display, an organic EL display, an LED display, a plasma display, any other display or a monitor known in this technical field.

The storage 129 stores a trained model generated in the first embodiment and the second embodiment. The storage 129 stores MR data arranged in k space by an image generation function 1413, image data generated by the image generation function 1413, etc. The storage 129 stores various types of examination protocols, conditions for imaging etc., including a plurality of imaging parameters that define examination protocols. The storage 129 stores programs corresponding to various functions performed by the processing circuitry 141. The storage 129 is, for example, a semiconductor memory element, such as a RAM and a flash memory, a hard disk drive, a solid state drive, or an optical disk. The storage 129 may be a drive or the like configured to read and write various types of information with respect to a portable storage medium such as a CD-ROM drive, a DVD drive, or a flash memory, etc.

The processing circuitry 141 includes, as hardware resources, a processor and a memory such as a read only memory (ROM) or a RAM not shown, and generally controls the MRI apparatus 2. The processing circuitry 141 includes the system control function 1411, the image generating function 1413, a grouping function 1415, and a calculation function 1417. These various functions are stored in the storage 129 in a form of programs executable by a computer. The processing circuitry 141 is a processor that reads programs corresponding to the various functions from the storage 129 and executes them to realize the functions corresponding to the programs. In other words, the processing circuitry 141 that has read the programs has, for example, the functions of the processing circuitry 141 shown in FIG. 12.

FIG. 1 illustrates that the aforementioned functions are implemented by the single processing circuitry 141; however, the processing circuitry 141 may be configured of a combination of a plurality of independent processors, and the functions may be implemented by the processors executing the respective programs. In other words, each of the aforementioned functions may be configured as a program, and single processing circuitry may execute each program, or each of the functions may be implemented in independent program-execution circuitry specific to respective functions.

The term “processor” used in the above description means, for example, a central processing unit (CPU), a graphics processing unit (GPU), or an application specific integrated circuit (ASIC), a programmable logic device ((e.g., a simple programmable logic device (SPLD), a complex programmable logic device (CPLD), and a field programmable gate array (FPGA)).

The processor realizes various functions by reading and executing programs stored in the storage 129. The programs may be directly integrated in a circuit of the processor, instead of being stored in the storage 129. In this case, the processor realizes functions by reading and executing programs which are integrated in the circuit. Note that the couch control circuitry 109, the transmitter 115, the receiver 119, and the sequence control circuitry 121 or the like are similarly configured by electronic circuitry such as the processor described above.

The processing circuitry 141 controls the MRI apparatus 2 by the system control function 1411. Specifically, the processing circuitry 141 reads the system control program stored in the storage 129, deploys it on the memory, and controls each circuitry of the MRI apparatus 2 in accordance with the deployed system control program. For example, the processing circuitry 141 reads an examination protocol from the storage 129 by the system control function 1411 based on an imaging condition input by the operator via the interface 125. The processing circuitry 141 may generate the examination protocol based on the imaging condition. The processing circuitry 141 transmits the examination protocol to the sequence control circuitry 121, and controls imaging on the subject P. The processing circuitry 141 arranges, by the image generation function 1413, MR data in a read out direction in the k space in accordance with, for example, the strength of the readout gradient magnetic field. The processing circuitry 141 performs the Fourier transform on the MR data filled in the k space to generate an MR image.

The processing circuitry 141 processes the generated MR image, and generates channel subsets, by the grouping function 1415 similar to the grouping function 133. For example, in the case of imaging that requires temporal resolution, such as dynamic MRI, a plurality of MR images are generated in time series on one slice. Therefore, the MR images are set as input channels for the trained model. The processing circuitry 141 groups a plurality of adjacent channels, which have a close physical relationship in the time series, and generates channel subsets.

The processing circuitry 141 applies, by the calculation function 1417 similar to the calculation function 135, the trained model to the channel subsets, so that the convolutional processing can be performed in units of channel subsets and output data can be obtained. The output data obtained after applying the trained model may be a denoised MR image or an image in which, for example, a tumor is segmented. Thus, the embodiment can be applied to any case in which the trained model such as the CNN is applicable to medical images.

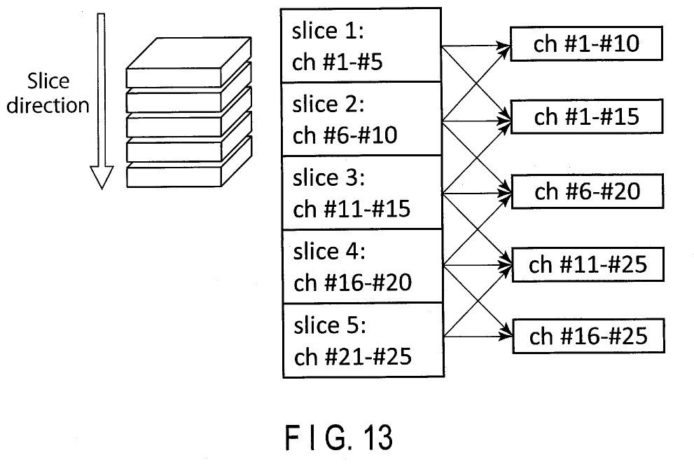

An example of generation of channel subsets in a case of using MR images as input data at the time of training or utilization will be explained with reference to FIG. 13.

FIG. 13 shows the correspondence between channels and MR images, namely slice images which are imaged in the slice direction.

For example, it is assumed that data of each MR image is set to five channels.

In this case, a first channel subset is generated by using slice 1 (channels #1 to #5) and slice 2 (channels #6 to #10). A second channel subset is generated by using slice 1, slice 2 and slice 3 (channels #11 to #15). Thus, the physical relationship of neighboring slices can be maintained.

In the case of dynamic MRI for performing multislice imaging, a plurality of MR images is generated in each slice. Therefore, there are two types of physical relationship between MR images: a time-series (time) and a spatial position. In this case, the channel subsets are generated in consideration of the two types of physical relationship.

The third embodiment described above is the MRI apparatus; however, the aforementioned processing is applicable to medical data acquired by another type of medical diagnosis apparatus. Specifically, the input data according to the present embodiment may be raw data collected by imaging a subject by a medical imaging apparatus, or medical image data generated by reconstructing the raw data. The medical imaging apparatus may be a single modality apparatus such as an MRI apparatus, an X-ray computed tomography apparatus (CT apparatus), an X-ray diagnostic apparatus, a positron emission tomography (PET) apparatus, a single photon emission CT apparatus (SPECT apparatus), and a ultrasound diagnostic apparatus, and also may be a combined modality apparatus such as a PET/CT apparatus, a SPECT/CT apparatus, a PET/MRI apparatus, and a SPECT/MRI apparatus.

The supply of the trained model to the MRI apparatus or any other medical imaging apparatus may be performed at any point in time between the manufacturing and the installation of the medical imaging apparatus in a medical facility, or at the time of maintenance.

The raw data of the embodiment is not limited to the original raw data collected by the medical imaging apparatus. For example, the raw data of the embodiment may be computational raw data generated by processing medical image data with forward projection processing. Alternatively, the raw data of the embodiment may be raw data obtained by processing original raw data with any signal processing, such as signal compression processing, resolution decomposition processing, signal interpolation processing, and resolution composite processing. Furthermore, if the raw data of the embodiment is three-dimensional raw data, it may be hybrid data obtained by restoration processing of only one axis or two axes. Similarly, the medical images of the embodiment are not limited to original medical images generated by a medical imaging apparatus. For example, the medical images of the embodiment may be medical images obtained by processing original medical images with any image processing, such as image compression processing, resolution decomposition processing, image interpolation processing, and resolution composite processing.

According to the third embodiment described above, a high quality image can be generated at a high speed by installing a trained model using the CNN into an MRI apparatus. When utilizing the trained model, if input data includes a medical image formed of a plurality of slices, a slice having a remote physical relationship (time or slice position) is not included in the channel subsets and therefore not used in the convolutional processing. Therefore, in the output obtained by the embodiment, an undesired artifact outside the neighboring slice images is prevented from occurring.

Furthermore, the functions described in connection with the above embodiments may be implemented, for example, by installing a program for executing the processing in a computer, such as a workstation, etc., and expanding said program in a memory. The program that causes the computer to execute the processing can be stored and distributed by means of a storage medium, such as a magnetic disk (a hard disk, etc.), an optical disk (CD-ROM, DVD, Blu-ray (registered trademark) etc.), and a semiconductor memory.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A data processing apparatus comprising processing circuitry configured to:
group a plurality of channels of input data based on a physical relationship between the input data to classify the plurality of channels into a plurality of subsets; and
perform convolutional processing of the input data in units of subsets for the plurality of subsets.

2. The apparatus according to claim 1, wherein the physical relationship is based on a physical quantity including at least one of data collection time or data spatial position.

3. The apparatus according to claim 2, wherein the processing circuitry groups the plurality of channels of the input data so that the physical quantity falls within a predetermined range.

4. The apparatus according to claim 1, wherein the input data is medical image data including a plurality of frames that are temporally continuous, and
the processing circuitry groups the plurality of channels in every adjacent frames.

5. The apparatus according to claim 1, wherein the input data is medical image data including a plurality of slices that are spatially continuous, and
the processing circuitry groups the plurality of channels in every adjacent slices.

6. The apparatus according to claim 1, wherein the processing circuitry performs the convolutional processing in a convolutional neural network.

7. The apparatus according to claim 1, wherein the processing circuitry is further configured to:
generate additional subsets, each including a combination of channels different from the channels of the plurality of subsets based on the physical relationship; and
perform the convolutional processing of the input data in units of the additional subsets.

8. The apparatus according to claim 1, wherein the processing circuitry performs the convolutional processing a plurality of times for one subset and outputs results of the convolutional processing of the respective times to a lower layer of network.

9. The apparatus according to claim 1, wherein if the plurality of channels includes a first data set including a first number of channels, and a second data set including a second number of channels different from the first number of channels and having a different data property from the first data set,
the processing circuitry groups channels selected from the first data set and the second data set based on the physical relationship to classify the grouped channel as one subset.

10. The apparatus according to claim 1, wherein the processing circuitry performs the convolutional processing for the subsets and all of the channels relating to the input data.

11. The apparatus according to claim 1, wherein the plurality of subsets includes a first subset including a first number of channels, and a second subset including a second number of channels, the second number being greater than the first number.

12. The apparatus according to claim 1, wherein the plurality of subsets includes a subset including a combination of channels discretely selected from the channels that satisfy conditions of the physical relationship.

13. A magnetic resonance imaging apparatus comprising processing circuitry configured to:
acquire a magnetic resonance (MR) signal;
generate a plurality of MR images from the MR signal;
group a plurality of channels corresponding to the MR images based on a physical relationship of the MR images to classify the plurality of channels into a plurality of subsets; and
apply a trained model to the MR images for correction, the trained model being for convolutional processing of the MR images in units of subsets for the plurality of subsets; and
output the corrected MR images.

14. A machine learning apparatus for training of a network model using training data, which is a set of input data and correct data corresponding to the input data, the apparatus comprising processing circuitry configured to:
group a plurality of channels of the input data based on a physical relationship between the input data to classify the plurality of channels into a plurality of subsets; and
perform convolutional processing of the input data in units of subsets for the plurality of subsets.
